(19) United States
(12) Patent Application Publication
(10) Pub. No.: US 2018/0035879 A1
(43) Pub. Date: Feb. 8, 2018

(54) AN ILLUMINATION DEVICE FOR ILLUMINATING CLOSE OBJECTS AND METHOD THEREOF

(71) Applicant: FORUS HEALTH PRIVATE LIMITED, Bangalore (IN)

(72) Inventors: Cothuru Santosh Kumar, Bangalore (IN); Shyam Vasudeva Rao, Bangalore (IN); Timothy Poston, Bangalore (IN); Anand S. Vinekar, Bangalore (IN)

(21) Appl. No.: 15/547,388

(22) PCT Filed: Jun. 4, 2015
(86) PCT No.: PCT/IB2015/054240

§ 371 Date: Jul. 28, 2017

(30) Foreign Application Priority Data
Jan. 30, 2015 (IN) 472/CHE/2015

(51) Int. Cl. A61B 1/06; A61B 1/07; A61B 1/227; A61B 1/00

(52) U.S. Cl. CPC A61B 1/0607; A61B 1/0019; A61B 1/0684; A61B 1/07; A61B 1/227; A61B 1/0676; A61B 1/00096; A61B 1/042

(57) ABSTRACT

Embodiments of the present disclosure provides an illumination device (300), comprises of one or more sources (301) which provides light arranged in a predefined configuration and a collar (303) with a first end (304) and a second end (305) to guide light received at the first end towards the second end. The collar guides the light in a medium between an internal wall and an external wall of the collar by reflection. The light from the second end is used to illuminate a target area for an imaging device. The method of illuminating a target area by an illumination device comprises of powering one or more sources to provide light for illumination and guiding the light in the medium to illuminate on the target area.

### TECHNICAL FIELD

The present disclosure relates to an illumination device. More particularly, the disclosure relates to an illumination device for illuminating a close object for performing an examination.

### BACKGROUND

It is often necessary, in using an optical device for close examination of an object, to shine strong light onto the object or for interior examination into the object. Typical optical devices are otoscopes, ophthalmoscopes, and endoscopes of many types according to the part of the body where the optical devices are inserted. A similar need for especially directed light occurs with microscopes, but since the apparatus typically surrounds the sample there are more options in providing that light, including trans-illumination from behind the sample. In examining an intact structure, and especially an interior one, with a single maneuverable device, the illumination must usually emerge from the device structure, near to the optical pathway along which light returns for study, at the distal end of the device (where the returning light enters it).

However, with most source types it is impractical to create bright light at the point where the light emerges. The principal reasons are well illustrated by the incandescent bulb, which is hard to miniaturize and very fragile when small, and by the nature of incandescence (light emission at high temperature) also yields heat, which can be painful or dangerous. These factors may change with newer technology such as light-emitting diodes and organic light-emitting diodes (LEDs and OLEDs), but the current art is to create light elsewhere, by an arc lamp or other intense source, filter out the infrared wavelengths (heat) and transfer the visible light by optical fiber to the emergence point. We illustrate this in FIG. 1, in a simplified representation of an otoscope 100 for examination of the ear.

The conventional otoscope 100 in FIG. 1 is configured with a bundle of optical fibers 104, for illuminating an object to be examined. The examination optics of the otoscope 100 consists of a convex lens 101 by which a clinician can look through the conical chamber 102 to see beyond tips 103, which may be narrower than shown, and may or may not include a transparent window or additional lens 105. (In other devices the lenses 101 and 105 may be replaced by a more complex light manipulation system, often culminating in a camera. Any system of manipulating the returning light, including adjustable lenses, mirrors, filters, etc., is compatible with this discussion of illumination management.) Light originates in a lamp 106, passes into a bundle 104 of optical fibers which spread out around the conical chamber 102, and emerges at the tips 103. Alternatively, a lamp that is larger and more intense (and produces more heat) than can fit easily into the hand-held part of a device may be coupled to it by a longer optical bundle 104, and placed in a static part of the system.

For clarity, FIG. 1 shows sharper curves in the fibers than are appropriate in an optimal design. High curvature leads to stress on the fibers, and leakage of light. The fiber placement must be very carefully designed to avoid these problems, and performed accurately in manufacture of each unit produced. This is a significant element in manufacturing cost.

Such configurations are very widely used in the current art, but they present an inherent problem that it is an objective of the present invention to address. Optical fiber for such uses typically has a diameter in the tens of micrometers, so that wave optics is more relevant to the behavior of the light than is the ray optic approximation. A bundle of geometric rays inside a fiber, glancing against the surface at an angle smaller than the critical angle θ_{c} (this angle θ_{c} is approximately equal to 40°, depending on materials) for total internal reflection, would mostly emerge from the tips 103 as rays within θ_{c} of a fiber axis: not precisely a beam, but somewhat directional. If the rays had crossed a substantial gap from the lamp 106 to the optical fibers 104, they would geometrically enter close to the fiber axis direction, diverging only if the optical fiber is significantly curved, to emerge as a tighter beam. However, on the scale of the tips 103, individual rays are a poor approximation to optics. Just as light passing through a small hole is diffracted in all directions (irrespective of the incident wave angle), the tips 103 act to a good approximation as a ring of isotropic point sources. This has strong implications for intensity of illumination.

Consider a circle {X²+Y²=1, Z=0} of isotropic emission points in the XY plane, such that an infinitesimal segment dθ at (X, Y, Z)=(cos θ, sin θ, 0) irradiates a point (x, y, z) with a intensity of

b(x, y, z)dθ=Idθ/((x−Y)²+(y−Y)²+(z−Z)²) (1)

This is perfectly general for a uniform thin ring of light sources similar to the tips 103, since we may choose its center as the origin and its radius as the unit of length. In a plane z=Z from the light source ring, the total intensity at a point p=(z, y, Z) is the integral of (1) from θ=0 to θ=2π. By symmetry this depends only on Z and the distance r=\sqrt{(x²+y²)} of p from the z-axis, so the integral reduces to that of

Idθ/((r−cos θ)²+(sin θ)²+Z²)=Idθ/(r²+1+Z²−2r cos θ) (2)

which is graphed in FIG. 2 for various Z. When Z=0.3 the brightness of illumination at distance r from the z-axis is shown by the curve 203, with sharp peaks 201 near r=±1. A flat white surface at 0.3 from the tips 103 would thus show a soft bright ring, of about the same radius. FIG. 2 shows the corresponding curves for distances increasing in steps of 0.1. The peaks are still visible in the curve 206 for a distance of 0.6, but flatten out in the curve 208 at distance 0.9, with the near-level region 207 giving a near-constant illumination disk on a flat surface at that distance. At a distance of 1.5 the curve 205 has a clear maximum 204, giving a soft bright spot on the corresponding surface.

These very specific non-uniform lighting patterns follow directly from the geometry of the situation, as long as the point sources are non-directional (as fiber-optic tips 103 approximately are). In the case of an ophthalmoscope or fundus camera, illumination through the cornea and lens of the eye changes the pattern, but the effect at the distance of the retina remains unadjustable: brightest in the central part of the image. Non-uniform lighting makes it harder to distinguish clinically important features.

### SUMMARY OF THE INVENTION

One or more shortcomings of the prior art are overcome and additional advantages are provided through the present disclosure. Additional features and advantages are realized through the techniques of the present disclosure. Other embodiments and aspects of the disclosure are described in detail herein and are considered a part of the claimed disclosure.

Embodiments of the present disclosure relate to an illumination device. The illumination device comprises one or more sources which provide light. The one or more sources are arranged in a predefined configuration. The illumination device also comprises a collar with a first end and a second end. The light from the one or more sources is received by the first end of the collar and guided by the collar towards the second end of the collar. The collar guides the light in a medium between an internal wall and an external wall of the collar, in our preferred embodiment by the principle of Total Internal Reflection (TIR). The light from the second end illuminates an object for close examination.

The method of illuminating a target area by an illumination device comprises powering one or more sources to provide light for illumination and guiding the light in the medium by reflection at the walls of the collar to illuminate the object.

The foregoing summary is illustrative only and is not intended to be in any way limiting. In addition to the illustrative aspects and features described above, further aspects, and features will become apparent by reference to the drawings and the following detailed description.

### BRIEF DESCRIPTION OF THE DRAWINGS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and with reference to the accompanying figures, in which:

FIG. 1 illustrates a conventional optical device, in accordance with prior art;

FIG. 2 illustrates light intensities from a conventional fiber-optic illumination ring at different distances;

FIG. 3A illustrates an embodiment of the illumination device in accordance with an embodiment of the present disclosure;

FIG. 3B illustrates another embodiment of the illumination device in accordance with an embodiment of the present disclosure;

FIG. 4 illustrates an example embodiment of the illumination device in accordance with an embodiment of the present disclosure;

FIG. 5 illustrates a cross-section view of another example embodiment illuminating device as an imaging device in accordance with an embodiment of the present disclosure.

It should be appreciated by those skilled in the art that any diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION OF THE INVENTION

The foregoing_has broadly outlined the features and technical advantages of the present disclosure in order that the detailed description of the disclosure that follows may be better understood. Additional features and advantages of the disclosure will be described hereinafter, which form the subject of the claims of the disclosure. It should be appreciated by those skilled in the art that the conception and specific aspect disclosed may be readily utilized as a basis for modifying or designing other structures for carrying out the same purposes of the present disclosure.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, a specific embodiment thereof has been shown by way of example in the drawings and will be described in detail below. It should be understood, however that it is not intended to limit the disclosure to the particular forms disclosed, but on the contrary, the disclosure is to cover all modifications, equivalents, and alternatives falling within the spirit and the scope of the disclosure.

The terms “comprises”, “comprising”, or any other variations thereof, are intended to cover a non-exclusive inclusion, such that a setup, device or method that comprises a list of components or steps does not include only those components or steps but may include other components or steps not expressly listed or inherent to such setup or device or method. In other words, the description of one or more elements in a system or apparatus proceeded by “comprises . . . a” does not, without more constraints, preclude the existence of other elements or additional elements in the system or apparatus.

Any embodiment of the present disclosure relates to an illumination device. The illumination device comprises one or more sources arranged in a predefined configuration to provide light, and a collar consisting of a first end and a second end, which guides the light towards the second end to illuminate an object. The light from the one or more sources is received by the first end of the collar and guided to the second end of the collar in a medium between an internal wall and an external wall of the collar, in a preferred embodiment by Total Internal Reflection (TIR), though other mechanisms of reflection may be substituted within the spirit of the present invention. The collar is made of a transparent medium. The TIR is achieved because of difference in the refractive indices of the medium composing the collar and the media surrounding the collar and substantially surrounded by it. The collar along with the first end and second end is designed to obtain a predefined pattern of illumination on the object to be illuminated. The method for illuminating the object with the help of the illumination device of the present disclosure involves powering of the sources to provide the light for illumination and guiding of the light through the collar to obtain the predefined pattern.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

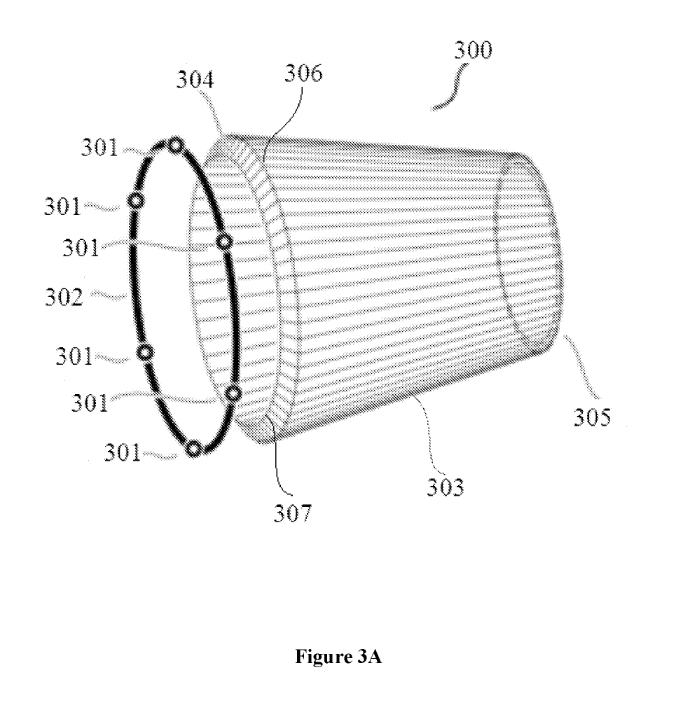

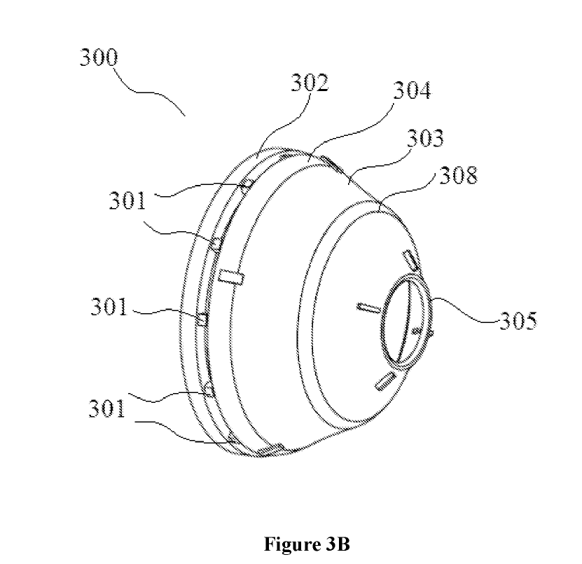

FIG. 3A illustrates an exemplary embodiment of the illumination device in accordance with an embodiment of the present disclosure.

As shown in FIG. 3A, the illuminating device 300 comprises one or more sources 301 and a collar 303. Each of the one or more sources 301 provides light, and these sources 301 are arranged in a predefined shape, which is a ring 302 in the instant embodiment. The inner and outer walls of the collar 303 are of a conical shape in the instant embodiment, having a first end 304 and a second end 305. In one embodiment, the predefined shape 302 of one or more sources is matched with the shape of the first end 304 of the collar 303. The light from the sources 301 is received at the first end 304 of the collar. The light received at the first end 304 is guided by the collar between an external wall 306 and the inner wall 307 by reflection at the walls 306 and 307, which in a preferred embodiment is caused by Total Internal Reflection (TIR), towards the second end 305 through the medium of the material of the collar 303. In a preferred embodiment, the medium outside the external wall 306 and the medium inside the inner wall 307 are each of low refractive index, each being one of vacuum and gaseous. The collar 303 is made up of a transparent material. The TIR of the light within the walls of the collar 306 and 307 is achieved because of the higher refractive index of the medium between the walls 306 and 307 of the collar 303 than in the media inside and outside the collar 303. The light emerging from the second end 305 is used to illuminate the object. The shape of the entry surface 307 and the exit surface 305, together with the shape of the walls 306 and 307 of the collar 300 in the embodiment helps to obtain the light at the second end 305 with a predefined pattern as required for illuminating the target area. In a preferred embodiment the exit surface 305 is smaller than the entry surface 307, giving rise to a substantially conical form for the collar, since it is often an operational requirement to have a small tip for the examining device (such as the devices 400 and 500 below) in which the illumination device 300 is embedded. Operational constraints on the entry surface 307 are often less, allowing more space in which to place the light sources 301 and their power supplies, and to dissipate heat.

FIG. 3B illustrates another embodiment of the illumination device in accordance with an embodiment of the present disclosure;

As shown in FIG. 3B, an illumination device 300 consists of a defined curve 308 in the collar 303. The sources 301 are arranged in a predefined shape 302. The predefined shape is matched with the shape of the first end 304 of the collar 303. The light from the sources 301 is received at the first end 304 and guided towards the second end 305 of the collar 303. In a preferred embodiment the light passing through the collar will undergo TIR, which is achieved because of the difference between the refractive index of the medium between the walls of the collar and the refractive indices of the media outside and surrounded by the walls of the collar as illustrated in FIG. 3A. The defined curved surface 308 in the collar 303 helps in obtaining a predefined pattern as required for illuminating the object.

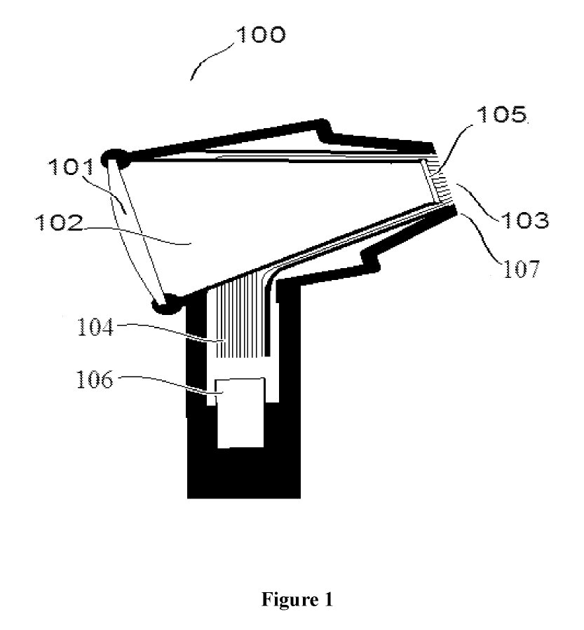

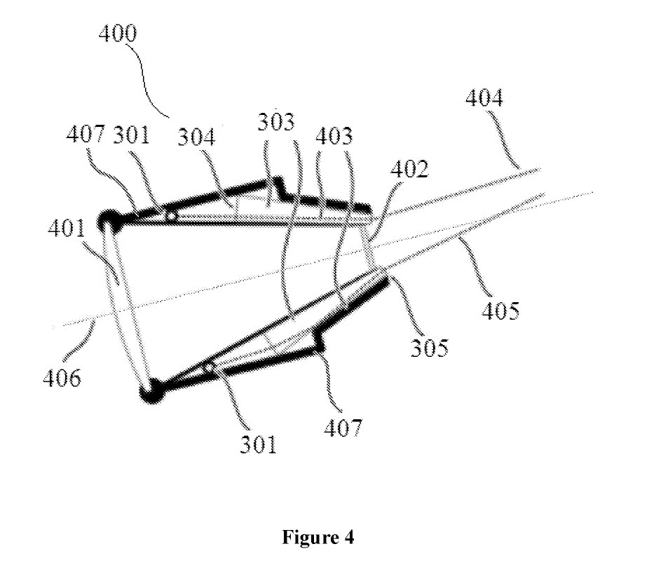

FIG. 4 illustrates an example embodiment of the illumination device as part of an imaging device in accordance with an embodiment of the present disclosure. In this example the imaging device is an otoscope, differing from the otoscope shown in FIG. 1 by the use of the present invention for illumination, rather than the optical fibers 104 common in current art. In other embodiments the imaging device is one of endoscopes, ophthalmoscopes, fundus cameras, microscopes, and other devices for examination of close objects. We use the terms ‘distal’ (farther from the user) and ‘proximal’ (nearer the user) in preference to ‘front’ and ‘rear’ which may be understood relative either to the user to or to the object examined. In some of our discussion the ‘first end’ is synonymous with ‘proximal end’, and ‘second’ with ‘distal’.

As shown in FIG. 4, the imaging device is symmetrical with respect to an optical axis 406, and consists of a front end lens 401, a rear end lens 402 and the illumination device. The lens 401 and 402 serve the purpose of examining an object illuminated by the illumination device. In one embodiment, an examiner examines the object which is present in the vicinity of the distal end lens 402 side through the proximal end lens 401. The sources 301 are arranged in the form of a ring with rotational symmetry about the optical axis 406. The predefined shape of arranging of the sources is matched with the shape of the first end 301 of the collar 303. The sources 301 may be one of a set of disjoint light emitting structures, a single circular light-emitter such as a loop of electroluminescent wire, the tips of optical fibers bringing light from elsewhere, and such other sources as will be evident to those skilled in the art. Further, the sources are placed in a position that is near to the proximal end of the collar 303 and also that avoids blocking of the light returned by the object to the imaging device. The light from the sources 301 is received at the first end 304 and guided through the collar 303 by reflection at the inner and outer walls towards the second end 305. This system of reflections guides of the light. In a preferred embodiment the reflections occur by total internal reflection, achieved because of the higher refractive index of the medium with respect to the refractive index of the media beyond the surfaces of the walls of the collar 303. Alternatively, the walls may be made of reflective material, in which case the medium of the collar is not required to have a refractive index conducive to total internal reflection. The medium must be transparent in the frequencies used by the imaging system and can be one of solid, liquid, gaseous and vacuum, subject to the requirements already discussed for its refractive index. Two rays of the light emitted at the second end are illustrated as 404 and 405 in FIG. 4. The combined intensity of all such rays helps in illuminating the object as required by an examiner to examine the object. The imaging device includes a support structure which holds the collar in a fixed spatial relation to the optical axis 406. Many configurations of such a support structure will be evident to those skilled in the art, and the walls 403 are shown with exemplary and non-limiting intent to illustrate a manner in which such support may be achieved. In this embodiment, the sources 301 are lodged in the walls 403, which may be considered as one of the parts of the imaging device rather than as a part of the illumination device. Alternatively, the sources may be mounted on a separate ring as a part of the illumination device which is illustrated as 302 in FIGS. 3A and 3B, or may be mounted directly on the collar 303, avoiding the need for separate support.

In embodiments where the light is generated at the sources 301, rather than generated elsewhere and transferred by the optical fiber, the imaging device must also provide support for power supplies to the sources 301. In our preferred embodiment this power is supplied by wires, but wireless power transfer would be within the spirit of the invention. Many configurations of support and power supply will be evident to those skilled in the art. A housing 407 is provided to the imaging device which helps in holding the lenses 401 and 402, the collar 303 and the sources 301 in place to construct the embodiment. A more even lighting pattern requires less total light to achieve adequate illumination across an imaging field, and consequently less power. Hence the sources 301 in the imaging device may be powered for short periods by batteries within the imaging device, without the need for an electrical cable connection to a base unit for powering, or an optical cable connection to a base unit for light. If the examiner looks through the device, or if an embedded camera connects wirelessly, the device can thus be hand held without any cable encumbrance.

Light from the sources 301 that enters the collar 303 may be approximated by a continuum of energy-carrying rays, refracted when they cross between the collar 303 and a medium such as air with a different refractive index. The space between the sources 301 and the surface of the first end of the collar 304, as shown in FIG. 3A, may be filled with air, or other material. The light emerging from the surface of the second end 305 may cross into a space which may be one of air, water, and optical gel according to the environment in which the imaging device is used. The refractive indices of the surfaces 304 and 305 and the surrounding space must be taken into account when designing any particular embodiment of an imaging device, since they modify the angles of refraction and hence the required geometry of the surfaces of the collar.

In any embodiment, a ray may travel like an exemplary ray 404, in a straight line between the first end 304 and the second surface 305. More typically, as exemplified by the ray 405, a ray may meet another surface on the walls of the collar and be reflected back into the wall, one or more times, before emerging from the collar guide 303. In a preferred embodiment the reflections occur because the ray meets the wall at an angle less than the critical angle for total internal reflection. A radial cross-section of the first end and second end surfaces 304 and 305 may be straight, as exemplified in FIG. 4, or curved, which adds a dispersive or concentrating effect on the rays passing through them. Defining the curves gives additional design parameters in specifying the three-dimensional form of a collar 303. In the case of a rotationally symmetric guide, an exemplary set of parameters to define its form can include specifying curves drawn as 304 and 305 as planar Bézier splines with eight parameters each and sixteen overall, joined by straight lines. If the curves 304 and 305 are assumed straight, only end points of 304 and 305 need to be specified, giving an eight-parameter space of possible designs. Further, the design parameters are of substantial range with a set of N number of parameters defining a collar shape as a collar design space (D).

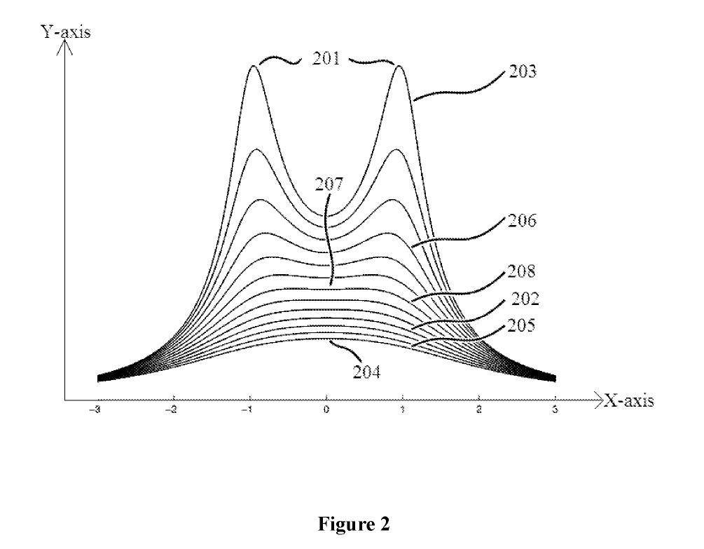

In an exemplary embodiment, most rays entering by the front end 304 emerge from the second end 305, though some may be lost by striking other surfaces at greater than a critical angle. The ray need not be restricted to a cross-sectional plane, as in the examples 404 and 405. The sources 301 may also emit out-of-plane rays and the rays which meet the first end 304 only can reflect zero or more times, moving around the optical axis 406, as they progress toward the second end 305. In any embodiment, just as in computing the curves is illustrated in FIG. 2, the oblique rays should be included in final illuminance values, so that a closed formula for the emitted illuminance pattern is not easily found. However, given a shape specification for the collar 303, numerical integration over all the rays from the sources 301 that meet the surface of the first end 304 is practical by means known to those skilled in the art. Thus search in the collar design space D is done for a best fit between the pattern of illumination produced by the collar and a desired pattern such as substantial uniformity on imaging field of the retina. In general exact uniformity is neither attainable, nor clinically necessary. We consider the imaging region considered to be illuminated ‘substantially’ uniformly, if the illumination as measured in lux does not vary by more than a factor of two between points in the field.

Similarly, a planar shape is referred as ‘substantially’ circular, if the radial distance of its points from a common center does not vary by more than a factor of two, thus excluding the case of a disk. A solid shape is ‘substantially’ conical if its cross-sections orthogonal to a common axis are substantially circular, with a radius decreasing from the first end 304 to the second end 305 of the collar 303. If to within a tolerance of 10% the decrease in cross-sectional radius is proportional to distance from the first end 304 that is the wider end, then it is referred to as ‘strongly conical’. If the shape can be described as two strongly conical shapes having different constants of proportionality, with the wider end of one abutting the narrower end of the other, then it is referred to as ‘bent conical’. If a shape is substantially conical with cross-sectional radius decreasing continuously but not proportionally with distance from the wider end, then it is referred to as curvilineal conical. Any collar design space D where designs are all conical, in one of the senses just described is within the spirit of the present invention, as is the use of any substantially conical shape for the light guide. Many methods for search within the collar design space D will be apparent to those skilled in the art, but options include trial and error, neural network training, genetic algorithms, and gradient ascent of a function quantifying agreement between achieved and desired patterns of illumination.

In one embodiment, other desired pattern of illumination other than uniform illumination may be used. The pattern of illuminated light allows computation of a shape of the object, or of a refractive geometry of the object such as a cornea between the imaging device and tissue reflecting the light. For example, the pattern of illumination may cast a bright ring on retina of the eye, creating in the imaging device a ring whose deviation from circularity reveals astigmatic distortion in cornea of the eye.

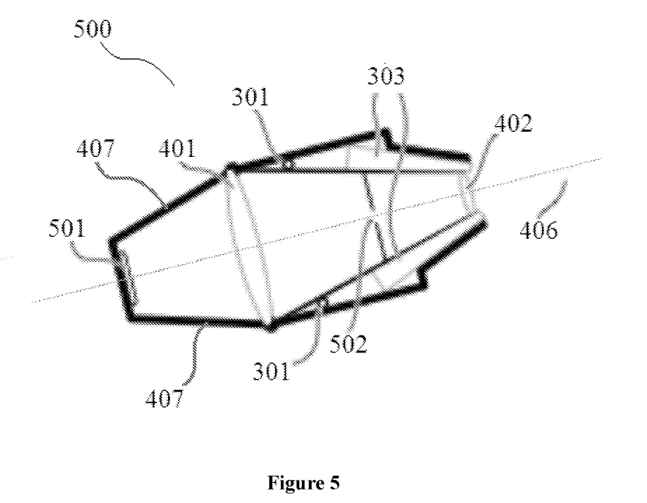

FIG. 5 illustrates a cross-section view of another example embodiment illumination device 500 in accordance with an embodiment of the present disclosure.

The illustration in FIG. 5 is for a simple digital camera, shown in cross-section through the optical axis 406. The lenses 401 and 402, in combination with the aperture 502 form, at the position of a digital sensor array 501, a real image of the objects that are distal to the distal lens 406, at a preferred range of distances. The object is illuminated by light from the sources 301 guided by the collar 303. Powering and controlling of the digital sensor array 501 is achieved with computers. The computers are configured to receive, modify and display the real image of the object. The embodiment may be configured to wired or wireless communication. The number of lenses 401 and 402 and configuration of lenses 401 and 402 is varied and is one of fixed in place or movable to modify the focus. The optical power of the lenses 401 and 402 may in an embodiment be varied by application of a DC voltage to modify the shape of a meniscus forming a surface of a lens. These and other camera configuration issues may all be varied within the spirit of this invention, whose concern is to provide controlled illumination on the objects which are present near the distal lens 402. The digital camera may be specialized for examination of the interior of an eye or ear, or any other object which is to be studied at close range. The shape of the collar 303, and the nature and configuration of the sources 301, may vary within the spirit of the invention as discussed above in relation to FIG. 5. A housing 407 is provided to the embodiment which helps in holding the lenses 401 and 402, the collar 303 and the sources 301 in place to construct the application 500 and also protect from external disturbances.

Our preferred embodiment, illustrated in FIG. 5, is to the case of a digital camera. In particular we apply it the case of a digital camera designed to examine the retina of infants born prematurely, of more complex design than the general camera schematic in FIG. 5. However, the invention is applicable wherever a device for imaging nearby objects requires illumination for the objects and this illumination must be provided by light accompanying the imaging device.

In addition, the illumination device may be used in various applications which require an efficient illumination of a near object. The illumination of the objects with the help of the illumination device can be obtained in the predetermined pattern as required for examining the object.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise.

The terms “a”, “an” and “the” mean “one or more”, unless expressly specified otherwise.

When a single device or article is described herein, it will be readily apparent that more than one device/article (whether or not they cooperate) may be used in place of a single device/article. Similarly, where more than one device or article is described herein (whether or not they cooperate), it will be readily apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include a separate device with components that contribute only to the functionality here disclosed.

The foregoing description of various embodiments and applications of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto. The above specification, examples and data provide a complete description of the manufacture and use of the composition of the invention. Since many embodiments of the invention can be made without departing from the spirit and scope of the invention, the invention resides in the claims hereinafter appended.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the disclosure of the embodiments of the invention is intended to be illustrative, but not limiting, of the scope of the invention, which is set forth in the following claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

In addition, where features or aspects of the disclosure are described in terms of Markush groups, those skilled in the art will recognize that the disclosure is also thereby described in terms of any individual member or subgroup of members of the Markush group.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

For those figures which are expository of the present invention, as distinct from the discussion of current art in FIGS. 1 and 2, we include the following key to the reference numbers used.

### REFERRAL NUMERALS

| Reference Number | Description |
|---|---|
| 300 | illumination device |
| 301 | sources |
| 302 | arrangement of the sources |
| 303 | collar |
| 304 | first or proximal end of the collar |
| 305 | second or distal end of the collar |
| 306 | external wall of the collar |
| 307 | internal wall of the collar |
| 308 | defined curve on the collar |
| 400 | application of the illumination device |
| 401 | proximal lens |
| 402 | distal lens |
| 406 | optical axis |
| 407 | housing |
| 500 | an example illumination device |
| 501 | digital camera |
| 502 | aperture |

1. A digital camera for examining a target area, comprising:
an illumination device, comprising:
one or more sources arranged in a predefined configuration, wherein each of the one or more sources provide light; and
a collar, with a first end and a second end, guides the light towards the second end to illuminate a target area, wherein the light from the one or more sources is received by the first end of the collar and guided to the second end of the collar by reflection in a medium between an internal wall and an external wall of the collar;
an imaging unit coupled with the illumination device for imaging the target area, comprising:
a pair of lenses, wherein each of the pair of lenses is associated with the first end and the second end of the collar to control focus of the target area; and
a digital sensor array placed at predefined distances with the pair of lenses, along an optical axis of a digital camera, to obtain a real image of the target area;
and
a housing configured to hold the illumination, device and the imaging unit with respect to the optical axis for examining the target area.

2. The digital camera as claimed in claim 1, wherein the collar is made of a transparent material.

3. The digital camera as claimed in claim 1, wherein the medium is one of gaseous and vacuum.

4. The digital camera as claimed in claim 1, wherein the medium has a higher refractive index compared to a refractive index of media beyond surfaces of the internal wall and the external wall of the collar.

5. The digital camera as claimed in claim 4, wherein the light received at the first end of the collar is guided by Total Internal Reflection (TIR) towards the second end.

6. The digital camera as claimed in claim 1, wherein each of the one or more sources is one of a Light Emitting Diode (LED) and an Organic LED (OLED).

7. The digital camera as claimed in claim 1, wherein the predefined configuration is a ring.

8. The digital camera as claimed in claim 1, wherein the predefined configuration is matched with a shape of the first end of the collar.

9. The digital camera as claimed in claim 8, wherein the shape of the collar is one of conical and curved shape with one or more curved surfaces formed by the internal wall and the external wall of the collar.

10. The digital camera as claimed in claim 8, wherein the shape of the collar between the first end and the second end and the shape of the first end and the second end, are defined to provide a predefined pattern of illumination on the target area.

11. The digital camera as claimed in claim 10, wherein the light guided by the collar is uniformly illuminated throughout the target area.

12. (canceled)

13. (canceled)

14. (canceled)

15. The digital camera as claimed in claim 1, wherein the imaging unit comprises an aperture placed between the pair of lenses along the optical axis to obtain the real image.

16. The digital camera as claimed in claim 1, wherein the pair of lenses are configured to be one of fixed and movable to control the focus.

17. The digital camera as claimed in claim 1, wherein the pair of lenses are applied with a Direct Current (DC) to vary optical power associated with the pair of lenses, the DC current modifies shape of meniscus associated with the pair of lenses.
